(19) United States
(12) Patent Application Publication
(10) Pub. No.: US 2002/0013532 A1
(43) Pub. Date: Jan. 31, 2002

(54) Method and system for imaging the interior of a body part and self-focusing endoscopic probe for use therein

(76) Inventors: Myron J. Czubko, Ann Arbor, MI (US); Harry McKinley, Southampton, MI (US); G. Neil Haven, Martinsville, IN (US)

Correspondence Address: David R. Syrowik, Brooks & Kushman P.C., 1000 Town Center, 22nd Floor, Southfield, MI, 48075-1351, US

(21) Appl. No.: 09/859,125

(22) Filed: May 16, 2001

Related U.S. Application Data
(60) Provisional application No. 60/204,563, filed on May 16, 2000.

(51) Int. Cl. A61B 6/00

(52) U.S. Cl. 600/478; 600/109; 600/160

(57) ABSTRACT

Methods and systems for imaging the interior of a body part and self-focusing endoscopic probes for use therein are provided. The self-focusing endoscopic probe includes an array of optical fibers for transmitting light to illuminate the interior of the body part to obtain a corresponding reflected light signal. A first mechanism is provided for preventing a first portion of the reflected light signal from passing through a reflection plane so that sufficient focus is maintained over a predefined focal range. A second mechanism allows a second portion of the reflected light signal to pass through the reflection plane. A rod lens or an array of optical fibers transmits the second portion of the reflected light signal to a detector plane to form an image of the interior of the body part in the detector plane. An optical element substantially collimates the second portion of the reflected light signal into a beam having a cross-sectional area of a substantially fixed dimension.

### CROSS-REFERENCE TO RELATED APPLICATION

This application claims the benefit of U.S. provisional application Ser. No. 60/204,563 entitled “System and Method for Endoscopic Dental Visualization” filed May 16, 2000.

### BACKGROUND OF THE INVENTION

1. Field of the Invention

This invention relates generally to methods and systems for imaging the interior of a body part and self-focusing endoscopic probe for use therein. This invention specifically relates to inspection and visualization methods and apparatus and, more particularly, to a family of endoscopic probes which are rapidly interchangeable and usable without any need for re-focusing adjustments. This family of devices is particularly useful in medical and dental applications where it is desirable to complete a medical procedure without the need to focus a visualization instrument in order to obtain an acceptable working image.

2. Background Art

Current art in the area of medical and dental visualization offers four general techniques:

Direct line-of-sight visualization.

The hand-held dental mirror. The dental mirror is used to allow visualization of dental surfaces not in the immediate line-of-sight of a dental technician or physician.

The dental microscope or loupe in conjunction with a dental mirror. The combination of microscope or loupe and mirror allows for the magnified visualization of dental surfaces both within the direct line-of-sight and outside the direct line-of-sight.

Various rigid rod-lens or fiber-optic systems. U.S. Pat. No. 5,429,502 discloses a rigid inter-oral camera with an integrated light source for viewing dental surfaces during dental procedures. U.S. Pat. No. 5,328,365 discloses a rigid endoscopic apparatus to be used for viewing and cleaning sub-gingival tooth surfaces. U.S. Pat. No. 4,790,751 discloses a fiber-optic method for the illumination of dental surfaces. U.S. Pat. No. 4,858,001 discloses a hand-held endoscopic apparatus with rotatable focal plane and an operative tip of small-enough diameter to be insertable into subgingival and inter-dental openings. Multiple lens-to-fiber and fiber-to-fiber interfaces are inherent in this system.

U.S. Pat. No. 4,184,175 discloses a system including afferent and efferent fiber-optic cables contained in a hand-held probe coupled to a light source and video camera, which is, in turn, connected to image display, recording, and processing means.

U.S. Pat. No. 4,423,436 describes an endoscope including afferent and efferent fiber optics coupled to a video camera.

Endoscopic probes utilizing fiber-optic image transmission have become commonplace within general medicine and dentistry, as well as other fields. U.S. Pat. No. 5,735,792 to Hoek et al. discloses a surgical instrument in which a fiber-optic assembly designed for spinal surgery containing both efferent light transmitting fibers connected to an illuminating means and afferent light transmitting fibers connected to an image viewing means is used for the illumination and visualization of body structures hidden from direct view.

U.S. Pat. No. 5,199,417 to Muller et al. discloses a semi-rigid endoscope with deflectable distal end designed for uteroscopy.

U.S. Pat. No. 4,947,245 to Ogawa et al. teaches an endoscopic imaging apparatus designed to be useful endodontically for the imaging of the root canal and oral cavity.

U.S. Pat. No. 5,328,365 to Jacoby describes an endoscopic device particularly useful for periodontal visualization and treatment.

Several patents in the prior art are directed to novel optical/mechanical means for allowing the operator of an endoscope to maintain or establish the focus of an instrument during a procedure.

U.S. Pat. No. 4,947,245 includes a description of a “Zooming Mechanism Adapter” meant to overcome limitations in view range and magnification of the endoscopic device.

U.S. Pat. No. 5,737,013 discloses a “Dental Video Camera with an Adjustable Iris” wherein an adjustable iris is adjusted between a nearly closed opening and a wide open opening in response to a user's focusing adjustment between a near field of focus and a far field of focus.

U.S. Pat. No. 6,019,721 to Holmes and Golay discloses a “Camera with Improved Focus Mechanism” in which a focus mechanism is taught that allows a lens element to be moved with respect to a stationary image sensor to achieve a range of focus. This patent also describes the state of the art of focus mechanisms for intra-oral cameras and endoscopes more generally.

In use, these and all other known endoscopes suffer from the requirement that re-focusing adjustments need to be made by the clinician during a medical or dental procedure when the standoff distance between the endoscopic probe and the field-of-view is changed, even minimally. In addition, if an endoscopic probe needs to be switched with another during a procedure (a not-infrequent occurrence in dental applications where probes of differing diameters may be required for insertion into, or viewing of, different dental surfaces) extensive refocusing may be necessary from one probe to another.

### SUMMARY OF THE INVENTION

An object of the present invention is to provide an improved method and system for imaging the interior of a body part and self-focusing endoscopic probe for use therein.

In carrying out the above objects and other objects of the present invention, a method for imaging an interior of a body part is provided. The method includes generating light and transmitting the generated light to illuminate the interior of the body part to obtain a corresponding reflected light signal. The method further includes preventing a first portion of the reflected light signal from passing through a reflection plane so that sufficient focus is maintained over a predefined focal range. A second portion of the reflected light signal is allowed to pass through the reflection plane. The second portion of the reflected light signal is transmitted to a detector plane to form an image of the interior of the body part in the detector plane. The method further includes measuring radiant energy in the image in the detector plane to produce a plurality of signals.

The step of transmitting the second portion of the reflected light signal may include the step of substantially collimating the second portion of the reflected light signal into a beam having a cross-sectional area of a substantially fixed dimension.

Further in carrying out the above objects and other objects of the present invention, a system for imaging an interior of a body part is provided. The system includes a light source for generating light and means for transmitting the generated light to illuminate the interior of the body part to obtain a corresponding reflected light signal. The system also includes means for preventing a first portion of the reflected light signal from passing through a reflection plane so that sufficient focus is maintained over a predefined focal range. The system further includes means for allowing a second portion of the reflected light signal to pass through the reflection plane. The system also includes means for transmitting the second portion of the reflected light signal to a detector plane to form an image of the interior of the body part in the detector plane, and means for measuring radiant energy in the image in the detector plane to produce a plurality of signals.

The means for transmitting the second portion of the reflected light signal may include an optical element for substantially collimating the second portion of the reflected light signal into a beam having a cross-sectional area of a substantially fixed dimension.

Still further in carrying out the above objects and other objects of the present invention, a self-focusing endoscopic probe is provided. The probe includes means for transmitting light to illuminate the interior of the body part to obtain a corresponding reflected light signal, and means for preventing a first portion of the reflected light signal from passing through a reflection plane so that sufficient focus is maintained over a predefined focal range. The probe also includes means for allowing a second portion of the reflected light signal to pass through the reflection plane. The probe further includes means for transmitting the second portion of the reflected light signal to a detector plane to form an image of the interior of the body part in the detector plane.

The means for transmitting the second portion of the reflected light signal may include an optical element for substantially collimating the second portion of the reflected light signal into a beam having a cross-sectional area of a substantially fixed dimension.

The means for transmitting light may include a light guide including an array of optical fibers.

The means for transmitting the second portion of the reflected light signal may include an array of optical fibers or a rod lens.

The probe may have a distal end and a single proximal end.

The probe may have a distal end and a pair of proximal ends.

The probe may have an arcuate, deflectable distal end.

The probe may also have a distal end wherein the probe further includes a housing mounted at the distal end and an object lensing subsystem including a plurality of lenses mounted in fixed relationship to one another and to the means for transmitting the second portion of the reflected light signal within the housing.

The means for allowing may be an aperture formed in the housing.

The means for allowing may further be an optical element within the housing.

The means for preventing may be an opaque material formed on one of the lenses.

The probe may further includes means for reducing undesired internal reflections within the housing.

The means for reducing may include an anti-reflective coating formed on the lenses.

The improvements offered through use of this invention over direct line-of-sight visualization include:

i) the ability to visualize occluded areas of the mouth; including, in one embodiment, the ability to visualize, subgingival, subcutaneous, and inter-dental structures,

ii) the ability to magnify areas of interest, and

iii) the ability to record areas of interest for later viewing.

The above object and other objects, features, and advantages of the present invention are readily apparent from the following detailed description of the best mode for carrying out the invention when taken in connection with the accompanying drawings.

### BRIEF DESCRIPTION OF THE DRAWINGS

FIG. 1 is a side schematic view of one embodiment of a probe constructed in accordance with the present invention with a combined afferent and efferent coupler;

FIG. 2 is a side schematic view of another embodiment of a probe constructed in accordance with the present invention with an afferent image transmission coupler and an efferent illumination coupler;

FIG. 3 is a cross-sectional view of an endoscopic shaft of the probe of FIG. 1;

FIG. 4 is a view similar to the view of FIG. 1 and illustrating the flexibility of the probe;

FIG. 5 is an enlarged view of a distal end of the probe of FIG. 1 with an apertured housing and with an object lensing subsystem indicated by phantom lines therein;

FIG. 6 is a schematic view illustrating a method and system of the present invention with the probe of FIG. 2; and

FIG. 7 is an enlarged view of a proximal end of the probe of FIG. 1 including suitable image digitization means adjacent the proximal end.

### DETAILED DESCRIPTION OF THE PREFERRED EMBODIMENTS

In general, the present invention discloses a novel and improved method and system and a family of endoscopes for use in medical and dental procedures wherein the need for re-focusing adjustments is obviated. Elimination of the need for re-focusing adjustments during medical/dental procedures results in more efficient and higher quality surgeries with less risk of contamination to the surgical site.

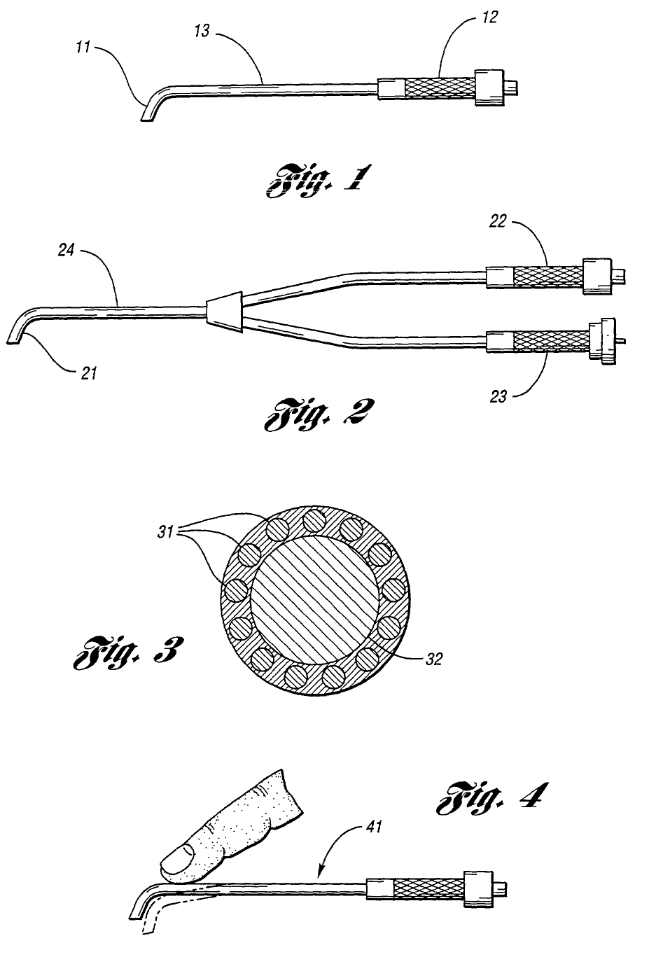

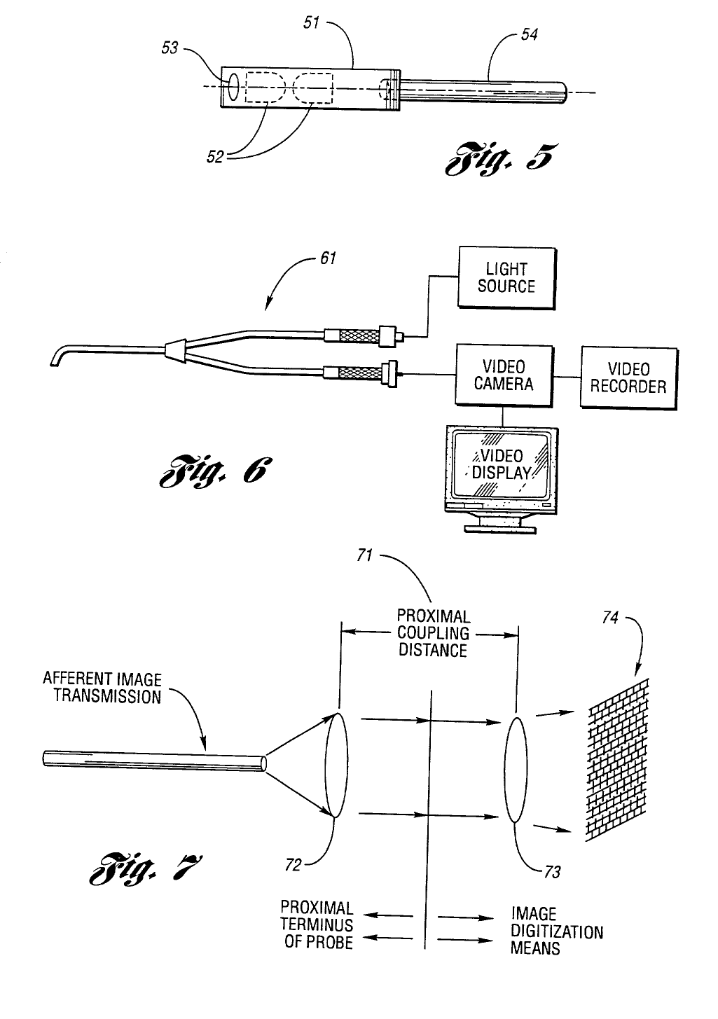

FIGS. 1 and 4 show an endoscopic probe (generally indicated at 41 in FIG. 4) incorporating the teachings of the present invention and which includes a distal end 11 and a proximal terminus 12. In a preferred embodiment, FIG. 4 shows an arcuate, deflectable distal terminus of the probe 41.

As shown in FIG. 3, between the termini 11 and 12, a rigid, semirigid, or flexible cylindrical shaft 13 extends which contains efferent light transmitting optical fibers 31 and an afferent means or mechanism 32 for transmitting an optical image from the distal (probe) end 11 to the proximal end 12. The afferent image transmission means 32 may be a coherent bundle of flexible fiber-optic strands or a rigid or semi-rigid rod lens.

FIGS. 1 and 4 show the endoscopic probe 41 with efferent and afferent optical fibers integrated into one proximal terminus 12. FIGS. 2 and 6 show an endoscope or probe, generally indicated at 61, having a distal end 21 and a pair of proximal termini 22 and 23. Between the termini 21 and 22 and 23, a cylindrical shaft 24, which may be rigid, semi-rigid or flexible, extends and contains afferent fibers and efferent fibers which have separate proximal termini. The diameter of the shaft 24 is 0.2 mm or greater depending on the number, size and type of efferent and afferent fibers or rod.

For example, a bundle measuring 0.7 mm constructed from 10,000 afferent image transmission fibers and approximately 20 efferent fibers for illumination with separate proximal termini may be purchased from Myriad Fiber Imaging of Massachusetts. The distal end 21 of the endoscopic probe 61 may be rigid or deflectable, arcuate or straight, or submillimeter diameter or larger.

At the distal end of both of the probes 41 and 61, an object lensing subsystem is mounted in a rigid cylindrical barrel housing 51. The subsystem includes a series of lenses 52 in fixed relation to one another. It is part of the innovation of the present invention that the objective lensing subsystem also includes an annular aperture 53 of dimension chosen to provide sufficient illumination for viewing, yet small enough to block light reflected from the illuminated body part at a reflection plane so that sufficient focus is maintained over the entire specified working range of the probe (41 or 61). This aperture 53 may take the form of a separate optical element in front of the optical train of the subsystem or anywhere within the train of optical elements. The aperture 53 may also take the form of an opaque annulus directly deposited onto one or more of the optical elements. An optical train designed for use with the afore described bundle of 10,000 afferent fibers is available from Liebman Optical of Easthampton, Mass. When used with an entrance aperture 53 of 80 microns, this optical train suffices to provide a resolution of 50 line pairs/mm over a focal range from 0.5 mm to 10 mm.

To reduce unwanted internal reflections in the distal end of the optical configuration, the lenses 52 receive an anti-reflective coating and the interior of the lens barrel 51 is optically blackened. The objective lensing subsystem is mated to the afferent means 32 of image transmission in a fixed geometric relation so that the focus of the lensing subsystem is fixed at a point intermediate between the specified working extremes of the endoscopic probe 41 or 61.

In a preferred embodiment of the present invention, the endoscopic probe 61 herein described is mated at its proximal end to an image digitization/processing/display/recording means and an illumination or light source such as a xenon source, visible laser source, or LED source, as shown in FIG. 6. Such imaging means are well known in the art.

In a preferred embodiment of the present invention, the image digitization means is a ⅓″ CCD camera such as is available from Sony Corp. incorporating means to electronically adapt to changing light conditions, thus removing from the user any need to adjust the illumination level during a medical/dental procedure.

The proximal ends 12 and 23 of the endoscopic probes 41 and 61, respectively, are fitted with an indexing coupler of a type well known to the art in order to minimize coupling geometry uncertainties between the proximal ends 12 and 23 and the image manipulation means or camera. Any residual non-repeatabilities in the mating geometry between the proximal ends 12 and 23 and the image manipulation means would, unabated, manifest themselves as a de-focusing of the image conveyed from the distal ends 11 and 21. It is part of the innovation of the present invention that an optical means is provided to abate the effect of the mechanical uncertainty in the proximal coupling distance 71 shown in FIG. 7. This optical means takes the form of a lensing system 72 formed as part of the proximal end of the afferent light path which acts to collimate the light forming the afferent image into a beam with a cross-sectional area of a fixed dimension which will remain constant across all the endoscopes or probes designed to be swappable within a given family of endoscopes. A corresponding optical means 73 at the input aperture of the image digitization means 74 serves to focus the collimated light onto a CCD array or other image digitization means. In this way, positional uncertainty in the proximal coupling distance 71 has no optical effect.

The innovations of the present invention are: (1) a mechanism such as a small annular aperture 53 at the distal probe terminus, and (2) a mechanism to collimate the afferent-image light at the proximal probe end (i.e., FIG. 7) in a standard manner. The innovations act together to determine a family of endoscopic probes which, when mated with appropriately-configured imaging means, may be freely swapped with one another no matter the other physical/optical characteristics of the individual endoscopic probes.

For instance, a 2 mm diameter probe designed to operate around a 5 cm focal distance with a magnification of twenty may be freely exchanged, as necessary, with a 0.7 mm diameter probe with a magnification of 200 designed to operate around a 5 mm focal distance. Moreover, these probes may be swapped without any intervening need to re-focus and they may be used entirely free from any need to re-focus.

In one embodiment, the diameter of the sheathed fiber-optic bundle is less than 1 mm to allow the insertion of the distal end of the bundle into subgingival, subcutaneous, or inter-dental areas. This allows the user invasively, inter alia, to:

search for cracks in the root canal wall,

look for narrowing of the canal,

find deviations of the canal or extra canals branching from the main canal,

search for holes out the side of the canal (created by mistakes in filing of the canal),

verify complete removal of the nerve,

find the end of the canal,

explore the depth and extent of dental caries,

observe the teeth under the gums for cracks or leaking under the edges of crowns,

explore sinus tracks or pathways of infection that lead from the end of an infected tooth to the gums,

generally aid invasive dental visualization for evaluative purposes, and

generally aid invasive dental visualization for repair purposes while looking at a video monitor.

In another embodiment, the diameter of the sheathed fiber-optic bundle is greater than 1 mm but less than 5 mm to afford the user a superior means of enhancing the visualization of dental, lingual, and gingival surfaces. This allows the user non-invasively, inter alia, to:

replace the dental mirror or microscope to look for cracks in the roots,

help cut the root-ends off and prepare the canal for a filing,

visualize between teeth and evaluate the contour of the bone there,

evaluate jaw fractures or find root tips that have broken off teeth during extraction,

generally aid in dental visualization for evaluative purposes, and

generally aid in dental visualization for repairs while looking at a video monitor.

While the best mode for carrying out the invention has been described in detail, those familiar with the art to which this invention relates will recognize various alternative designs and embodiments for practicing the invention as defined by the following claims.

What is claimed is:

1. A method for imaging an interior of a body part, the method comprising:
generating light;
transmitting the generated light to illuminate the interior of the body part to obtain a corresponding reflected light signal;
preventing a first portion of the reflected light signal from passing through a reflection plane so that sufficient focus is maintained over a predefined focal range;
allowing a second portion of the reflected light signal to pass through the reflection plane;
transmitting the second portion of the reflected light signal to a detector plane to form an image of the interior of the body part in the detector plane; and
measuring radiant energy in the image in the detector plane to produce a plurality of signals.

2. The method as claimed in claim 1 wherein the step of transmitting the second portion of the reflected light signal includes the step of substantially collimating the second portion of the reflected light signal into a beam having a cross-sectional area of a substantially fixed dimension.

3. A system for imaging an interior of a body part, the system comprising:
a light source for generating light;
means for transmitting the generated light to illuminate the interior of the body part to obtain a corresponding reflected light signal;
means for preventing a first portion of the reflected light signal from passing through a reflection plane so that sufficient focus is maintained over a predefined focal range;
means for allowing a second portion of the reflected light signal to pass through the reflection plane;
means for transmitting the second portion of the reflected light signal to a detector plane to form an image of the interior of the body part in the detector plane; and
means for measuring radiant energy in the image in the detector plane to produce a plurality of signals.

4. The system as claimed in claim 3 wherein the means for transmitting the second portion of the reflected light signal includes an optical element for substantially collimating the second portion of the reflected light signal into a beam having a cross-sectional area of a substantially fixed dimension.

5. A self-focusing endoscopic probe comprising:
means for transmitting light to illuminate an interior of a body part to obtain a corresponding reflected light signal;
means for preventing a first portion of the reflected light signal from passing through a reflection plane so that sufficient focus is maintained over a predefined focal range;
means for allowing a second portion of the reflected light signal to pass through the reflection plane; and
means for transmitting the second portion of the reflected light signal to a detector plane to form an image of the interior of the body part in the detector plane.

6. The probe as claimed in claim 5 wherein the means for transmitting the second portion of the reflected light signal includes an optical element for substantially collimating the second portion of the reflected light signal into a beam having a cross-sectional area of a substantially fixed dimension.

7. The probe as claimed in claim 5 wherein the means for transmitting light includes a light guide including an array of optical fibers.

8. The probe as claimed in claim 5 wherein the means for transmitting the second portion of the reflected light signal includes an array of optical fibers.

9. The probe as claimed in claim 5 wherein the means for transmitting the second portion of the reflected light signal includes a rod lens.

10. The probe as claimed in claim 5 wherein the probe has a distal end and a single proximal end.

11. The probe as claimed in claim 5 wherein the probe has a distal end and a pair of proximal ends.

12. The probe as claimed in claim 5 wherein the probe has an arcuate, deflectable distal end.

13. The probe as claimed in claim 5 wherein the probe has a distal end and wherein the probe further comprises a housing mounted at the distal end and an object lensing subsystem including a plurality of lenses mounted in fixed relationship to one another and to the means for transmitting the second portion of the reflected light signal within the housing.

14. The probe as claimed in claim 13 wherein the means for allowing is an aperture formed in the housing.

15. The probe as claimed in claim 13 wherein the means for allowing is an optical element within the housing.

16. The probe as claimed in claim 13 wherein the means for preventing is an opaque material formed on one of the lenses.

17. The probe as claimed in claim 13 further comprising means for reducing undesired internal reflections within the housing.

18. The probe as claimed in claim 17 wherein the means for reducing includes an anti-reflective coating formed on the lenses.
